(19) United States
(12) Patent Application Publication
(10) Pub. No.: US 2005/0226364 A1
(43) Pub. Date: Oct. 13, 2005

(54) Rotational computed tomography system and method

(75) Inventors: Bruno Kristiaan Bernard De Man, Clifton Park, NY (US); Samit Kumar Basu, Niskayuna, NY (US); Peter Michael Edic, Albany, NY (US); William Robert Ross, Scotia, NY (US); Mark Ernest Vermilyea, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/816,015

(22) Filed: Mar. 31, 2004

Related U.S. Application Data
(60) Provisional application No. 60/525,606, filed on Nov. 26, 2003.

(51) Int. Cl. G21K 1/12; H05G 1/60; A61B 6/00; G01N 23/00

(52) U.S. Cl. 378/9

(57) ABSTRACT

Geometries and configurations are provided for CT systems in which rotational loading is reduced, permitting higher speeds and lighter structures to be implemented in the systems. In certain embodiments a distributed and addressable rotating radiation source is provided with a rotating detector. In other embodiments a distributed and addressable stationary radiation source is provided with a rotating detector. In yet other embodiments a distributed and addressable radiation source is provided that rotates with respect to a stationary detector. The sources may be ring-like, arcuate and/or lines extending at least in the Z-direction. Sources may include a large number of distributed emitters arranged in lines, arcs and one- or two-dimensional arrays.

### BACKGROUND OF THE INVENTION

The present invention relates generally to the field of computed tomography imaging systems. In particular, the invention relates to geometries and configurations for sources and detectors in such systems designed to reduce the rotational load and to enhance speed and imaging abilities of the systems.

Computed tomography (CT) imaging systems have been developed in the past decades and now are prolific in medical diagnostics and other contexts. In general, such systems typically include an X-ray source, such as a conventional X-ray tube, positioned in a diametrically opposed location from a digital detector. The source and detector rotate on a gantry, and the source is triggered repeatedly or is on continuously during rotation to produce beams of X-ray radiation that are directed through a subject of interest and fall onto the detector on the opposite side of a gantry. The emitted radiation is attenuated by features and structures of the subject, and the transmitted radiation is measured by the detector. The measurements are usually converted to attenuation measurements and the resulting measurement data is then processed for reconstruction of useful images, typically presented as slices through the subject. Many such images may be produced in a single imaging sequence.

CT systems have proven extremely useful in producing excellent images of internal features of variety of subjects, including human and animal patients in a medical diagnostic context, internal configurations, components of parts and parcels, and so forth. Moreover, image reconstruction techniques have been continuously developed and refined to enhance the quality of such images. Current systems are available to operate in a variety of modes, capable of producing large volumes of data from which useful images can be reconstructed.

Conventional CT systems are not, however, without drawbacks. For example, to improve the temporal resolution of the resulting reconstructed images, the systems are rotated at increasingly high speeds. To balance the load of the X-ray source, detector, and associated circuitry and components, the gantry and support structures must be carefully designed and balanced. Moreover, the X-ray source and detector must be powered during operation, and the data must be extracted from the X-ray detector continuously. All of the elements, furthermore, undergo significant heating, requiring extraction of thermal energy during operation. These various challenges pose extremely difficult problems for system designers and those called upon to maintain the systems. Moreover, the sheer mass of the source, detector, and associated circuitry and components ultimately limits the rate of rotation of the gantry, and thereby limits the rate and number of view frames that can be collected in a unit of time.

There is a continuing need, therefore, for improvements in CT imaging systems that can facilitate rotation of the required system components for collection of useful measurement data. There is, at present, a particular need for improved system designs that permit more data to be collected per unit of time, or that would permit faster scan times so as to avoid artifacts and other problems associated with organ motion such as for the heart or even slight patient movements. In addition, there is a need for systems that allow acquiring data that is more mathematically complete and therefore allows reconstructing a large 3D volume while limiting cone beam reconstruction artifacts.

### SUMMARY OF THE INVENTION

The present invention provides novel CT configurations and geometries designed to respond to such needs. While presently contemplated applications for the systems include medical diagnostic imaging applications, the new geometries and configurations may find applications well outside the medical diagnostics context, including for part inspection, parcel and package handling and screening, baggage scanning, and so forth. In general, the configurations of the present invention reduce rotation loads of conventional CT systems while maintaining or even improving the quantity and quality of the measurement data. The configurations may include arrangements in which both a source and a detector are rotated, or may call for a rotation of only the detector, or only the source. In certain arrangements of the present technique, ring-like sources or ring-like detectors are employed that may be completely stationary within the system. The present technique is also based upon the provision of distributed X-ray sources that comprise multiple, independently addressable X-ray emitters. In other configurations, the sources are addressable in logical groups, for example pairs or triplets of emitters may be wired together. Unique configurations for these sources are provided that enable the various geometries and configurations. For example, the distributed X-ray sources may form a two-dimensional array. In other configurations the sources form rings around the imaging volume, partial rings around the volume, and lines along the “Z-direction” to use the conventional CT nomenclature. Moreover, the sources and detectors may be comprised of linear or planar sections respectively, which approximate the configurations discussed below.

Benefits of the invention flow from the significant reduction in the mass required for rotation. That is, for arrangements where the source of X-ray radiation is stationary, only the detector needs be rotated. Conversely, where the detector is stationary, only the distributed X-ray source needs be rotated. Higher rotational speeds may thus be attained with a lighter structure.

### BRIEF DESCRIPTION OF THE DRAWINGS

FIG. 1 is a diagrammatical representation of an exemplary CT system in accordance with aspects of the present technique;

FIG. 2 is a diagrammatical representation of an exemplary distributed source for use with a system of the type illustrated in FIG. 1;

FIG. 3 is a diagrammatical representation of a portion of detector for use with the system illustrated in FIG. 1;

FIG. 4 is a diagrammatical representation of a first exemplary CT system configuration, including a distributed source and a partial ring detector;

FIG. 5 is a diagrammatical representation of a further configuration, including a stationary distributed ring source with a rotational detector;

FIG. 6 is a diagrammatical representation of a further configuration, including a pair of stationary distributed ring sources with one or more rotating detectors;

FIG. 7 is a diagrammatical representation of a further configuration with a two-dimensional stationary distributed ring source and one or more rotating detectors;

FIG. 8 is a diagrammatical representation of a further configuration with a stationary ring detector used in conjunction with a line source along the Z-axis;

FIG. 9 is a diagrammatical representation of a further configuration with a stationary ring detector used in conjunction with an arc source, and one or more line sources in the Z-direction;

FIG. 10 is a diagrammatical representation of a further configuration with a stationary ring detector and one or more arc sources and one or more line sources in the Z-direction; and

FIG. 11 is a diagrammatical representation of a further configuration with a stationary ring detector and a two-dimensional array distributed source.

### DETAILED DESCRIPTION OF SPECIFIC EMBODIMENTS

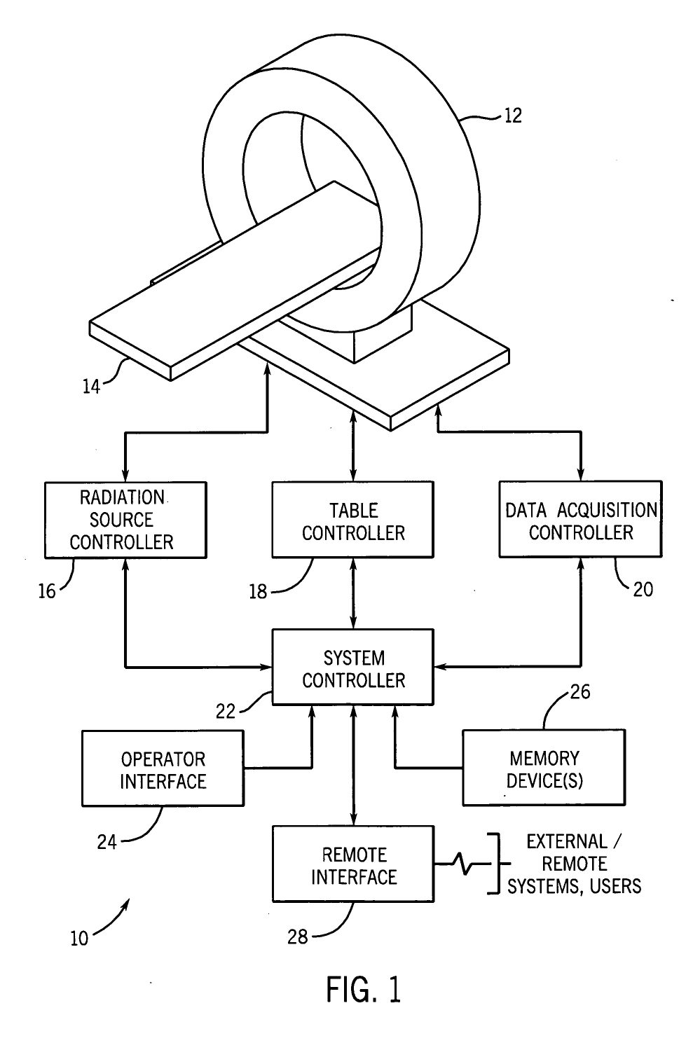

Turning now to the drawings, referring first to FIG. 1, a computed tomography (CT) system is illustrated and designated generally by reference numeral 10. The CT system 10 comprises a scanner 12 formed of a support structure and internally containing one or more stationary or rotational, distributed sources of X-ray radiation (not shown in FIG. 1) and one or more stationary or rotational digital detectors (not shown in FIG. 1), as described in greater detail below. The scanner is configured to receive a table 14 or other support for a patient, or, more generally, a subject to be scanned. The table can be moved through an aperture in the scanner to appropriately position the subject in an imaging volume or plane scanned during imaging sequences.

The system further includes a radiation source controller 16, a table controller 18 and a data acquisition controller 20, which may all function under the direction of a system controller 22. The radiation source controller 16 regulates timing for discharges of X-ray radiation which is directed from points around the scanner 12 toward a detector element on an opposite side thereof, as discussed below. In the present stationary CT arrangements, the radiation source controller 16 may trigger one or more emitters in a distributed X-ray source at each instant in time for creating multiple projections or frames of measured data. In certain arrangements, for example, the X-ray radiation source controller 16 may trigger emission of radiation in sequences so as to collect adjacent or non-adjacent frames of measured data around the scanner. Many such frames may be collected in an examination sequence, and data acquisition controller 20, coupled to detector elements as described below receives, signals from the detector elements and processes the signals for storage and later image reconstruction. In configurations described below in which one or more sources are rotational, source controller 16 may also direct rotation of a gantry on which the distributed source or sources are mounted. Table controller 18, then, serves to appropriately position the table and subject in a plane in which the radiation is emitted, or, in the present context, or generally within a volume to be imaged. The table may be displaced between imaging sequences or during certain imaging sequences, depending upon the imaging protocol employed. Moreover, in configurations described below in which one or more detectors or detector segments are rotational, data acquisition controller 20 may also direct rotation of a gantry on which the detector or detectors are mounted.

System controller 22 generally regulates the operation of the radiation source controller 16, the table controller 18 and the data acquisition controller 20. The system controller 22 may thus cause radiation source controller 16 to trigger emission of X-ray radiation, as well as to coordinate such emissions during imaging sequences defined by the system controller. The system controller may also regulate movement of the table in coordination with such emission so as to collect measurement data corresponding to volumes of particular interest, or in various modes of imaging, such as helical modes. Moreover, system controller 22 coordinates rotation of a gantry on which either the source(s), detector(s), or both are mounted. The system controller 22 also receives data acquired by data acquisition controller 20 and coordinates storage and processing of the data.

It should be borne in mind that the controllers, and indeed various circuitry described herein, may be defined by hardware circuitry, firmware or software. The particular protocols for imaging sequences, for example, will generally be defined by code executed by the system controllers. Moreover, initial processing, conditioning, filtering, and other operations required on the measurement data acquired by the scanner may be performed in one or more of the components depicted in FIG. 1. For example, as described below, detector elements will produce analog signals representative of depletion of a charge in photodiodes positioned at locations corresponding to pixels of the data acquisition detector. Such analog signals are converted to digital signals by electronics within the scanner, and are transmitted to data acquisition controller 20. Partial processing may occur at this point, and the signals are ultimately transmitted to the system controller for further filtering and processing.

System controller 22 is also coupled to an operator interface 24 and to one or more memory devices 26. The operator interface may be integral with the system controller, and will generally include an operator workstation for initiating imaging sequences, controlling such sequences, and manipulating measurement data acquired during imaging sequences. The memory devices 26 may be local to the imaging system, or may be partially or completely remote from the system. Thus, imaging devices 26 may include local, magnetic or optical memory, or local or remote repositories for measured data for reconstruction. Moreover, the memory devices may be configured to receive raw, partially processed or fully processed measurement data for reconstruction.

System controller 22 or operator interface 24, or any remote systems and workstations, may include software for image processing and reconstruction. As will be appreciated by those skilled in the art, such processing of CT measurement data may be performed by a number of mathematical algorithms and techniques. For example, conventional filtered back-projection techniques may be used to process and reconstruct the data acquired by the imaging system. Other techniques, and techniques used in conjunction with filtered back-projection may also be employed. A remote interface 28 may be included in the system for transmitting data from the imaging system to such remote processing stations or memory devices.

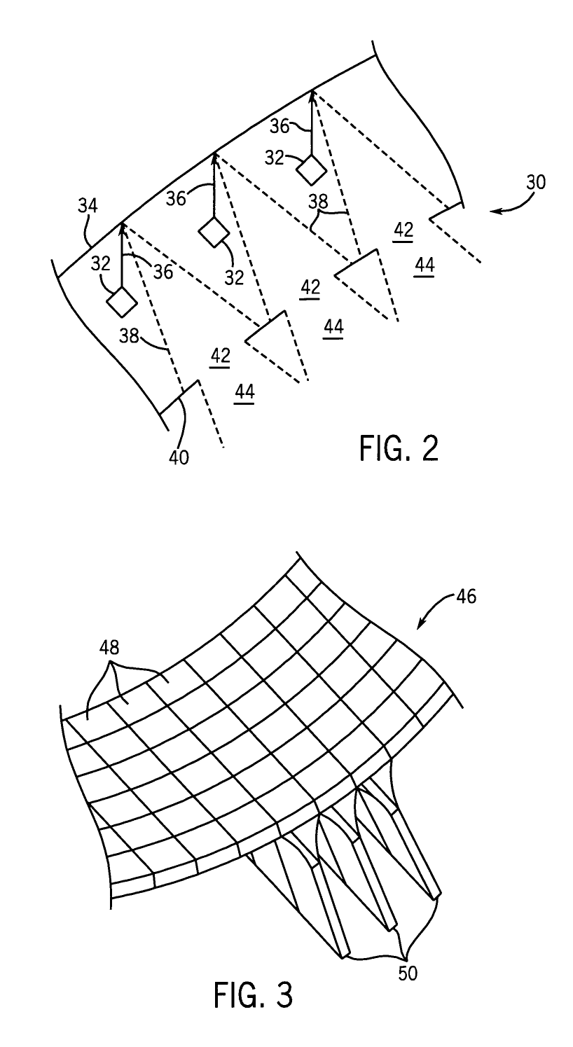

The scanner 12 of CT system 10 preferably includes one or more rotating or stationary distributed X-ray sources as well as one or more rotational or stationary digital detectors for receiving radiation and processing corresponding signals to produce measurement data. FIG. 2 illustrates a portion of an exemplary distributed X-ray source of the type that may be employed in the CT system. As shown in FIG. 2, in an exemplary implementation, the distributed X-ray source 30 may include a series of electron beam emitters 32 that are coupled to radiation source controller 16 shown in FIG. 1, and are triggered by the source controller during operation of the scanner. The electron beam emitters 32 are positioned adjacent to a target 34. Upon triggering by the source controller, the electron beam emitters 32 may emit electron beams 36 toward target 34. The target 34, which may, for example, be a tungsten rail or element, emits X-ray radiation, as indicated at reference numeral 38, upon impact of the electron beams. In reflection mode, X-rays are meant to be produced primarily on the same side of the target as where the electrons impact. In transmission mode, X-rays are produced at the opposite side of the target. The X-ray beams 38 are directed, then, toward a collimator 40, which is generally opaque to the X-ray radiation, but which includes openings or apertures 42. The apertures 42 may be fixed in dimension, or may be adjustable. Apertures 42 permit a portion of the X-ray beams to penetrate through the collimator to form collimated beams 44 that will be directed to the imaging volume of the scanner, through the subject of interest, and that will impact detector elements on an opposite side of the scanner.

A number of alternative configurations for emitters or distributed sources may, of course, be envisaged. Moreover, the individual X-ray sources in the distributed source may emit various types and shapes of X-ray beams. These may include, for example, fan-shaped beams, cone-shaped beams, and beams of various cross-sectional geometries. Similarly, the various components comprising the distributed X-ray source may also vary. In one embodiment, for example, a cold cathode emitter is envisaged which will be housed in a vacuum housing. A stationary anode is then disposed in the housing and spaced apart from the emitter. This type of arrangement generally corresponds to the diagrammatical illustration of FIG. 2. Other materials, configurations, and principals of operations may, of course, be employed for the distributed source. The emission devices may be one of many available electron emission devices, for example, thermionic emitters, carbon-based emitters, photo emitters, ferroelectric emitters, laser diodes, monolithic semiconductors, etc.

As discussed in greater detail below, the present CT techniques are based upon use of a plurality of distributed and addressable sources of X-ray radiation. Moreover, the distributed sources of radiation may be associated in single unitary enclosures or tubes or in a plurality of tubes designed to operate in cooperation. Certain of the source configurations described below are arcuate or ring-like in shape so as to positionable about the aperture in the scanner. Other sources are linear in configuration, so as to extend along the imaging volume, in the “Z-direction” in terms of the conventional CT nomenclature. The individual sources are addressable independently and separately so that radiation can be triggered from each of the sources at points in time during the imaging sequence as defined by the imaging protocol. Where desired, more than one such source may be triggered concurrently at any instant in time, or the sources may be triggered in specific sequences to mimic rotation of a gantry, or in any desired sequence around the imaging of volume or plane.

A plurality of detector elements form one or more detectors, which receive the radiation emitted by the distributed sources. FIG. 3 illustrates a portion of a detector which may be employed for the present purposes. The detector arrangement may be generally similar to detectors used in conventional rotational CT systems, but is preferably extended around a greater portion or the entire inner surface of the scanner in certain embodiments. Each detector may be comprised of detector elements with varying resolution to satisfy a particular imaging application. Particular configurations for the detector or detectors are summarized below. In general, however, the detector 46 includes a series of detector elements 48 and associated signal processing circuitry 50. These detector elements may be of one, two or more sizes, resulting in different spatial resolution characteristics in different portions of the measured data. Each detector element includes an array of photodiodes and associated thin film transistors. X-ray radiation impacting the detectors is converted to lower energy photons by a scintillator and these photons impact the photodiodes. A charge maintained across the photodiodes is thus depleted, and the transistors may be controlled to recharge the photodiodes and thus measure the depletion of the charge. By sequentially measuring the charge depletion in the various photodiodes, each of which corresponds to a pixel in the collected data for each acquisition, data is collected that indirectly encodes radiation attenuation at each of the detector pixel locations. This data is processed by the signal processing circuitry 50, which will generally convert the analog depletion signals to digital values, perform any necessary filtering, and transmit the acquired data to processing circuitry of the imaging system as described above.

A large number of detector elements 48 may be associated in the detector so as to define many rows and columns of pixels. As described below, the detector configurations of the present technique position detector elements across from independently addressable distributed X-ray sources so as to permit a large number of views to be collected for image reconstruction. Although the detector is described in terms of a scintillator-based energy-integrating device, direct conversion, photon counting, or energy discriminating detectors are equally suitable.

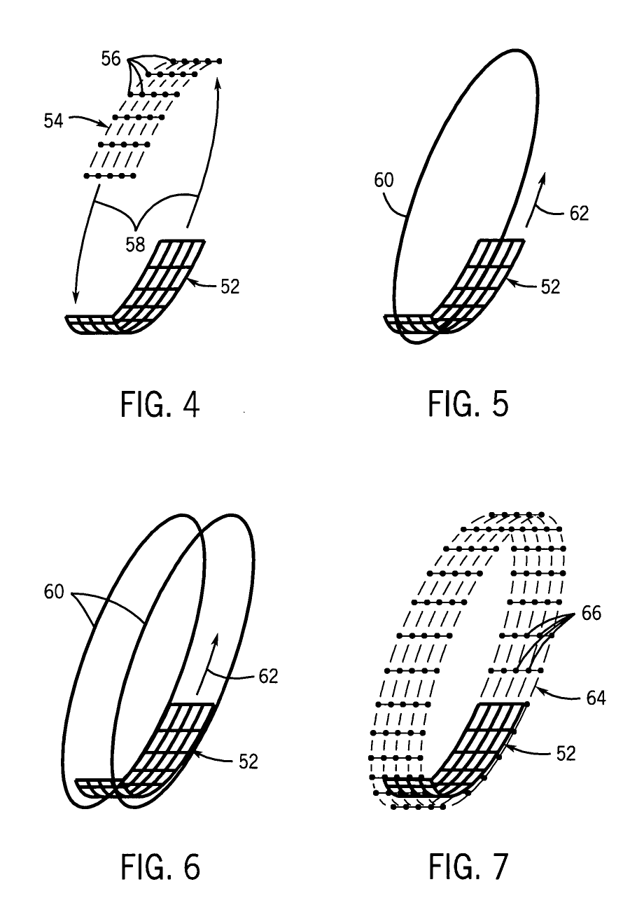

As will be appreciated by those skilled in the art, reconstruction techniques in CT systems vary in their use of acquired data, and in their techniques and assumptions for image reconstruction. It has been found, in the present technique, that a number of geometries are available for high-speed and efficient operation of a CT system, which provide excellent mathematical completeness of measured data for accurate image reconstruction while significantly reducing the rotational load on the CT scanner, particularly on the gantry and support structures. FIGS. 4-11 illustrate exemplary geometries and configurations for distributed sources and for detectors, certain of which are stationary in the CT scanner, but that can be used with conventional or improved image processing and image reconstruction algorithms.

As noted above, enhancement the present CT system configurations is attained by reduction of the rotational load on the system. In particular, presently contemplated embodiments employing distributed X-ray sources and ring or partial ring detectors are illustrated in FIGS. 4 through 11. In general, the arrangements are based upon certain preferred source and detector configurations. By way of example, a distributed source may include a plurality of independently addressable emitters arranged in an array extending at least partially around the circumference of the imaging volume and extending along the Z-axis (generally perpendicular to the imaging plane). Other source configurations may include lines of emitters along the Z-direction, arcuate sources having a plurality of emitters in a row extending around a portion of the circumference of the scanner, and complete ring sources extending substantially completely around the arcuate of the scanner. Detector configurations may be somewhat similar. That is, presently contemplated detector configurations for the improved CT system geometries may be generally similar to existing detectors in construction, but extend around a portion of the scanner aperture or completely around the aperture in a ring-like arrangement. In the description that follows, a ring source or ring detector refers to either a one-dimensional or two-dimensional array of source or detector elements, respectively, centered about some possibly arbitrary axis.

FIG. 4 illustrates a first exemplary embodiment of a reduced mass rotational CT system comprising a partial ring detector 52 and a distributed partial ring source 54, positioned both around the aperture of the scanner and along the Z-direction, that is, generally perpendicular to the plane of the scanner. However, the extent of the array source may be limited to a single arc source. Detector 52 may generally be of conventional construction, including a plurality of detector elements and associated circuitry of the type described above. Distributed partial ring source 54 is mounted with the detector 52 on a gantry for a rotation. The distributed partial ring source 54 may include a series of emitters 56 designed to be independently and separately addressable so as to emit X-ray radiation upon demand as described above. Both the detector and source are rotated during operation as indicated by arrows 58 in FIG. 4.

FIGS. 5, 6 and 7 illustrate embodiments in which a source is completely stationary, and the detector alone rotates by relatively conventional means, such as on a gantry. As shown in FIG. 5, a distributed ring source 60 may be employed which may have a plurality of emitters similar to the arrangement illustrated in FIG. 4 for distributed partial ring source 54. The emitters, again, are independently and separately addressable so as to permit emission of X-ray radiation in specific sequences as partial ring detector 52 rotates around the scanner aperture on a conventional gantry. Arrow 62 indicates the rotation of the partial ring detector 52 in this manner. FIG. 6 illustrates a similar arrangement in which a pair of distributed ring sources 60, are employed. The ring sources preferably flank the partial ring detector 52, which again rotates in the scanner, such as on a conventional gantry. Similarly, FIG. 7 illustrates a distributed ring array source 64 comprising a number of emitters 66 positioned both around the aperture of the scanner and along the Z-direction, that is, generally perpendicular to the plane of the scanner. In this embodiment, as well, a partial ring detector 52 is provided that rotates on a conventional gantry. The emitters 66 of the distributed ring array source 64 are independently and separately addressable so as to emit X-ray radiation toward the detector for imaging as the detector rotates. As will be appreciated by those skilled in the art, because the sources illustrated in FIGS. 5, 6, and 7 do not rotate, the gantry and other support structures may be considerably lighter than in conventional systems, and the detector may be rotated at higher speeds. Similarly, because the sources illustrated in FIGS. 4, 6, and 7 are distributed in along the Z-axis, the data completeness may be significantly higher than in conventional CT systems.

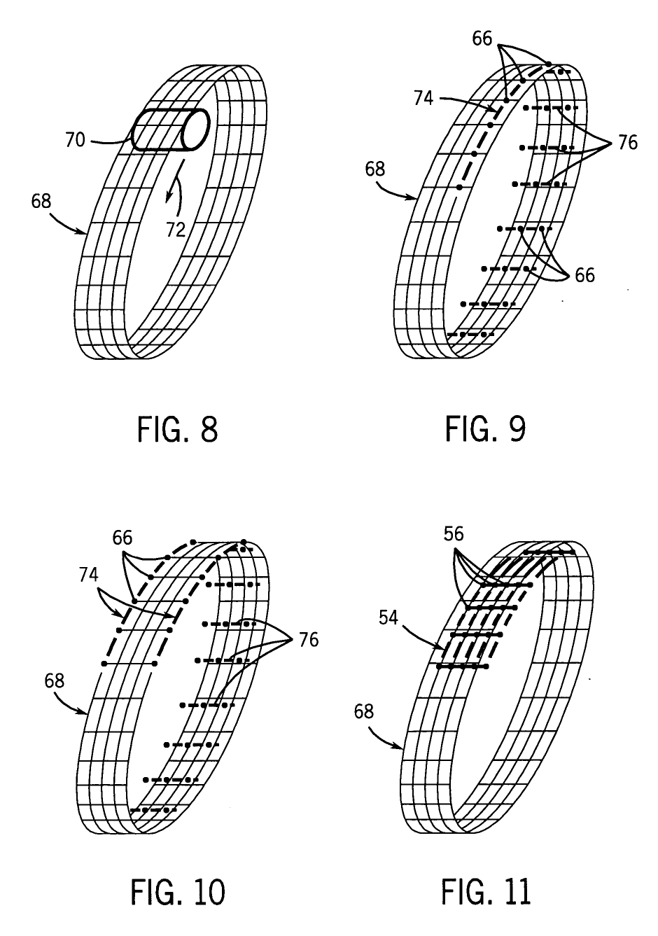

FIGS. 8, 9, 10 and 11 represent alternative configurations in which the detector or detectors are completely stationary within the CT scanner, and one or more distributed sources are rotated. In the arrangement of FIG. 8, for example, a ring detector 68 comprises a number of detector elements of the type described above, and completely encircles the scanner aperture. A line source in the Z-direction, indicated by reference numeral 70, is rotated in the system as indicated by arrow 72. The source 70 may be of the type where the target is a hollow cylinder rotating around its axis. The source 70 may also be of the type where the target consists of a number of segments of a disk that are offset in the Z-direction. The source 70 may also include a plurality of independently and separately addressable emitters, such that X-ray emissions may be generated toward locations on the detector 68 generally opposed to the location of the source. In this arrangement, only the source 70 need be rotated, leading to a significant reduction in the rotational load and requirements for support structures.

In the configuration of FIG. 9, a ring detector 68 of the type described with respect to FIG. 8 is employed, but with a combination of two types of distributed sources. The first distributed source, which may be termed a distributed arc source 74, extends partially around the scanner aperture along the inside surface of the ring detector 68. One or more distributed line sources 76 extend generally along the Z-direction and function in cooperation with the distributed arc source to generate radiation for imaging. The distributed arc source 74 and the distributed line sources 76 would be mounted on a conventional gantry for rotation within the ring detector 68. Similarly, FIG. 10 illustrates an arrangement including a pair of distributed arc sources 74, flanking the ring detector 68, and used in conjunction with one or more distributed line sources 76. Finally, in the arrangement of FIG. 11, a ring detector 68 is employed along with a distributed partial ring source 54 of the type discussed above with reference to FIG. 4. The distributed partial ring source 54 is rotated about the inner periphery of the ring detector to generate radiation for imaging.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. An imaging system comprising:
one or more distributed X-ray sources configured to generate X-ray radiation towards an imaging volume; and
one or more detectors for receiving the X-ray radiation after attenuation in the imaging volume and processing corresponding signals to produce measurement data,
wherein the distributed X-ray sources and/or the detectors are arranged about a scanner aperture such that at least one of the X-ray sources or detectors rotate in relation to the imaging volume during an imaging sequence.

2. The imaging system of claim 1 wherein the one or more distributed X-ray sources comprises at least one stationary distributed source positioned about a scanner aperture and the one or more detectors comprises at least one distributed detector configured to rotate around a scanner aperture.

3. The imaging system of claim 2 wherein the one or more distributed X-ray sources includes one or more two-dimensional arrays of source elements extending substantially around the aperture.

4. The imaging system of claim 2 wherein the one or more distributed X-ray sources includes one or more two-dimensional arrays of source elements extending around a portion of the aperture.

5. The imaging system of claim 2 wherein the one or more distributed X-ray sources includes one or more one-dimensional arrays of source elements extending substantially around the aperture.

6. The imaging system of claim 5 further comprising:
one one-dimensional array of source elements extending substantially around the aperture; and
one or more line sources.

7. The imaging system of claim 5 further comprising:
two or more one-dimensional arrays of source elements extending substantially around the aperture; and
one or more line sources.

8. The imaging system of claim 2 wherein the one or more distributed X-ray sources includes one or more one-dimensional arrays of source elements extending around a portion of the aperture.

9. The imaging system of claim 2 wherein the at least one distributed detector includes one or more two-dimensional arrays of detector elements extending around at least a portion of the aperture.

10. The imaging system of claim 2 wherein the at least one distributed detector includes one or more one-dimensional arrays of detector elements extending around at least a portion of the aperture.

11. The imaging system of claim 2 wherein the one or more distributed X-ray sources includes one or more line sources.

12. The imaging system of claim 1 wherein the one or more distributed X-ray sources includes a plurality of independently addressable source elements arranged in one or more arrays.

13. The imaging system of claim 1 wherein the one or more distributed X-ray sources comprises:
a cold cathode emitter housed in a vacuum housing; and,
a stationary anode disposed in a vacuum housing and spaced apart from the cold cathode emitter.

14. The imaging system of claim 1 wherein the one or more distributed X-ray sources comprises addressable emission devices and the emission devices comprises thermionic emitters, cold-cathode emitters, carbon-based emitters, photo emitters, ferroelectric emitters, laser diodes or monolithic semiconductors.

15. The imaging system of claim 1 wherein the one or more distributed X-ray sources comprises at least one distributed source configured to rotate around the scanner aperture and the one or more detectors comprises at least one stationary and distributed detector positioned about the scanner aperture.

16. The imaging system of claim 15 wherein the at least one distributed source includes one or more two-dimensional arrays of source elements.

17. The imaging system of claim 15 wherein the at least one distributed source includes one or more one-dimensional arrays of source elements.

18. The imaging system of claim 15 wherein the one or more one-dimensional arrays of source elements extend around at least a portion of the aperture.

19. The imaging system of claim 18 further comprising a one-dimensional array of source elements extending around at least a portion of the aperture, and one or more line sources.

20. The imaging system of claim 18 further comprising two or more one-dimensional arrays of source elements extending around at least a portion of the aperture and one or more line sources.

21. The imaging system of claim 17 wherein the at least one of the one or more one-dimensional arrays of source elements include at least one line source extending at least along a Z-direction.

22. The imaging system of claim 21, wherein the at least one line source comprises a target configured as a hollow cylinder rotating around its axis.

23. The imaging system of claim 15 wherein the at least one stationary and distributed detector includes one or more two-dimensional arrays of detector elements extending substantially around the aperture.

24. The imaging system of claim 15 wherein the at least one stationary and distributed detector includes one or more two-dimensional arrays of detector elements extending around a portion of the aperture.

25. The imaging system of claim 15 wherein the at least one stationary and distributed detector includes one or more one-dimensional arrays of detector elements extending substantially around the aperture.

26. The imaging system of claim 15 wherein the at least one stationary and distributed detector includes one or more one-dimensional arrays of detector elements extending around a portion of the aperture.

27. The imaging system of claim 1 wherein the one or more distributed X-ray sources comprises at least one distributed source configured to rotate around the scanner aperture and the one or more detectors comprises at least one distributed detector configured to rotate around a scanner aperture.

28. The imaging system of claim 27 wherein the at least one distributed source includes one or more two-dimensional arrays of source elements.

29. The imaging system of claim 27 wherein the at least one distributed source includes one or more one-dimensional arrays of source elements.

30. The imaging system of claim 27 wherein the one or more one-dimensional array of source elements extend around at least a portion of the aperture.

31. The system of claim 30 further comprising a one-dimensional array of source elements and one or more line sources.

32. The system of claim 30 further comprising two or more one-dimensional arrays of source elements and one or more line sources.

33. The imaging system of claim 29 wherein at least one of the one or more one-dimensional arrays of source elements includes at least one line source extending at least along a Z-direction.

34. The imaging system of claim 33, wherein the at least one line source comprises a target configured as a hollow cylinder rotating around its axis.

35. The imaging system of claim 27 wherein the at least one distributed detector includes one or more two-dimensional arrays of detector elements extending around at least a portion of the aperture.

36. The imaging system of claim 27 wherein the at least one distributed detector includes one or more one-dimensional arrays of detector elements extending around at least a portion of the aperture.

37. An X-ray imaging system for scanning a volume to be imaged, the system comprising:
one or more distributed X-ray sources substantially surrounding an imaging volume and configured to emanate an X-ray radiation;
a control circuit operably coupled to the distributed X-ray sources;
one or more detectors for receiving the X-ray radiation after attenuation in the imaging volume;
a motor controller configured to displace at least one of the distributed X-ray sources, and the detectors;
a processing circuit operably coupled to the detectors configured to receive the plurality of projection images and to form one or more reconstructed slices representative of the volume being imaged; and
an operator workstation operably coupled to the processing circuit configured to display the one or more reconstructed slices,
wherein the distributed X-ray sources and/or the detectors are arranged about a scanner aperture such that at least one of the X-ray sources or detectors rotate in relation to the imaging volume during an imaging sequence.

38. The X-ray imaging system of claim 37 wherein the one or more distributed X-ray sources comprises at least one stationary distributed source positioned about a scanner aperture and the one or more detectors comprises at least one distributed detector configured to rotate around a scanner aperture.

39. The X-ray imaging system of claim 37 wherein the one or more distributed X-ray sources comprises at least one distributed source configured to rotate around the scanner aperture and the one or more detectors comprises at least one distributed detector configured to rotate around a scanner aperture.

40. The X-ray imaging system of claim 37 wherein the one or more distributed X-ray sources comprises at least one distributed source configured to rotate around the scanner aperture and the one or more detectors comprises at least one stationary and distributed detector positioned about the scanner aperture.

41. A method of scanning a volume to be imaged, the method comprising:
providing one or more distributed X-ray sources for generating X-ray radiation towards an imaging volume; and
providing one or more detectors for receiving the X-ray radiation after attenuation,
wherein generating and receiving the X-ray radiation is accomplished by rotating at least one of the distributed X-ray sources or detectors in relation to the imaging volume during an imaging sequence.

42. The method of claim 41 wherein providing the one or more distributed X-ray sources comprises providing at least one stationary distributed source positioned about a scanner aperture and providing the one or more detectors comprises providing at least one distributed detector configured to rotate around a scanner aperture.

43. The method of claim 41 wherein providing the one or more distributed X-ray sources comprises providing at least one distributed source configured to rotate around the scanner aperture and providing the one or more detectors comprises providing at least one distributed detector configured to rotate around a scanner aperture.

44. The method of claim 41 wherein providing the one or more distributed X-ray sources comprises providing at least one distributed source configured to rotate around the scanner aperture and providing the one or more detectors comprises providing at least one stationary and distributed detector positioned about the scanner aperture.
